(19) United States
(12) Patent Application Publication
(10) Pub. No.: US 2015/0211064 A1
(43) Pub. Date: Jul. 30, 2015

(54) ASSAY AND METHOD FOR THE IDENTIFICATION OF INDIVIDUAL RESPONSIVENESS TO IMMUNOGLOBULIN THERAPY

(71) Applicants: Stefan Meuer, Heidelberg (DE); Thomas Geise, Heidelberg (DE); Christian Jacobi, Heidelberg (DE); Stefan Haag, Lachen (CH); Juergen Roemisch, Vienna (AT)

(72) Inventors: Stefan Meuer, Heidelberg (DE); Thomas Geise, Heidelberg (DE); Christian Jacobi, Heidelberg (DE); Stefan Haag, Lachen (CH); Juergen Roemisch, Vienna (AT)

(73) Assignee: OCTAPHARMA AG, Lachen (CH)

(21) Appl. No.: 14/114,276

(22) PCT Filed: Jun. 6, 2012
(86) PCT No.: PCT/EP2012/060699

§ 371 Date: Oct. 9, 2014

(30) Foreign Application Priority Data
Jun. 7, 2011 (EP) PCT/EP2011/059405

(51) Int. Cl. C12Q 1/68

(52) U.S. Cl. CPC C12Q 1/6883; C12Q 2600/156; C12Q 2600/112; C12Q 2600/118

(57) ABSTRACT

A method for determining the likelihood of response of an individual, suffering from a disease, towards immunoglobulin therapy has the steps of
providing a sample containing B- and T-lymphocytes, natural killer cells, invariant T-cells and monocytes of the individual;
genotyping of at least one of the polynucleotides of an ADAMTS9-Intron; a KLHDC8A-Intron or of a flanking region of the CD14 gene, and
awarding the value of 1 for the homozygous Single Nucleotide Polymorphism combinations, which suggests that the blood sample stems from a person which will not respond to immunoglobulin treatment,
while awarding the value of 0 for SNP not meeting that criteria, which suggests that the blood sample stems from a person which will respond to immunoglobulin treatment.

The invention pertains to a method of determining the individual responsiveness toward immunoglobulin therapy.

B cells identify pathogens when antibodies on their surface (B-cell receptor) bind to a specific foreign antigen. In response, B cell divide and differentiate into plasma cells, which secrete millions of copies of the antibody that recognize the activating antigen. Antibodies (also known as immunoglobulins) recognise targets comprising many different compounds, structures and those also as part of cellular structures and often neutralise their biological effect. The immune complexes are cleared fast leading to the elimination of a “target” molecule, accordingly, recognition, binding and removal function is doubtless of essential importance, which is substantiated by the fact that patients lacking (or have reduced) immunoglobulin levels are prone to serious and recurrent infections. Beyond this protection function towards intruders, immunoglobulins bear important regulatory function in balancing and regulating the immune system. Immunoglobulin products, usually derived from pooled blood or plasma donations and prepared according processes well known to the expert, are used for the treatment of IMID (immune mediated inflammatory diseases) and so called AID (autoimmune diseases), while those definitions may express identical or overlapping features of a disease. The immunoglobulin G (IgG) concentrates are usually applied intravenously (IVIG) or subcutaneously (SCIG), but may be also intramuscularly, inhaled, intra-ocularly, orally or topically. When B cells react aggressively against self, the potential for pathology is extreme. It is therefore not surprising that B-cell depletion is seen as an attractive therapy in patients with autoimmune diseases or immune mediated inflammatory diseases (IMIDs).

Natural killer (NK) cells are large granular lymphocytes that belong to the innate immune system because unlike T- or B-lymphocytes of the adaptive or antigen-specific immune system, NK cells do not rearrange T-cell receptor or immunoglobulin genes from their germline configuration. NK cells have been characterized as a lymphocyte lineage with both cytotoxic and cytokine-producing effector functions. NK cells accomplish selective lysis of cells on the basis of activating and inhibitory receptors relatively specific to the NK cell lineage. Activating receptors such as NKG2D recognize natural stress signal ligands or pathogen-derived ligands expressed on transformed or infected cells. In contrast, the inhibitory receptors of the CD94-NKG2A complex, KIR family (Killer-cell Immunoblobulin-like Receptors) bind self but not allogeneic MHC I (Major Histocompatibility Complex I). The KIR receptors are highly polymorphic, heterogeneously expressed among NK cells, and important in NK cell self-tolerance. During licensing, NK cells acquire functional competence following productive interactions between inhibitory receptors and self-MHC during development. Licensed NK cells express at least one of several possible inhibitory receptor alleles. Hence, MHC-recognition among NK cells is diverse so that at least one NK cell subset will respond to downregulation of any single MHC class I molecule. When activating NK cell effector functions are primed, IFN-γ is secreted and granzyme and perforin release are enhanced. NK cells also express Fcγ receptors, which recognize several IgG subclasses, to mediate antibody-dependent cellular cytotoxicity. These receptors and their roles in NK cell surveillance and cytolysis have been well studied and are closely associated with the identity of NK cells. Upon flow cytometry so called CD56^{bright} and CD56^{dim} can be differentiated which do or do not express Fcγ RIII (CD16). The interpretation of the flow cytometry results upon ‘gatings’ is well known to the skilled person.

Accordingly, 4 major sub-populations can be defined, which are

### CD16+/CD56^{bright}

### CD16+/CD56^{dim}

### CD16−/CD56^{bright}

### CD16−/CD56^{dim}

NK cells are an important part of the host defence. However, if dysregulated, e.g. as reason or in the course of diseases, they can direct themselves against “self-structures” with significant pathophysiological consequences, like attack of organ structures and oligodendrocytes in the periphery and brain. Although the mechanisms are not fully understood leading to such self-attacks, regaining control of these cells and reducing their killing power to a physiologically reasonable level is required. As killing efficiency is tightly connected to degranulation and release of effector molecules, it is an aim to control such degranulation to reduce damage.

The effectivity of intravenous immunoglobulin (IVIG) in autoimmune diseases was first described in the 1950ies and then in the 1980ies, when it was used to treat patients suffering from idiopathic thrombocytopenic purpura. In the mean-time many clinical studies demonstrated a beneficial effect of IVIG in autoimmune diseases. Among others, IVIG therapy is proven in Guillain-Barre syndrome (GBS), Kawasaki syndrome, chronic inflammatory demyelinising polyneuropathy, myasthenia gravis and corticosteroid-resistant dermatomyositis (Ephrem et al., 2005; Kazatchkine and Kaveri, 2001; Boros et al., 2005). A positive effect of IVIG on disease progression relapse rate and MRI enhancing lesions in multiple sclerosis (MS) was demonstrated by clinical studies as well (Sorensen PS, 2003; Sorensen et al., 2002). An established method to assess the status of disease is the EDSS Expanded Disability Status Scale (EDSS).

The complete mechanism of action of IVIG is still unclear but seems to involve the modulation of expression and function Fcγ receptors, interference with complement activation, modulation of T- and B-cell activation, -differentiator and -effector functions (Ephrem et al., 2005; Kazatchkine and Kaveri, 2001; Boros et al., 2005).

While immunoglobulin prophylaxis and treatment is successfully used in many patients, among them are cases who poorly or even not respond to immunoglobin application. Recent approaches suggest to qualitatively or quantitatively determine distinct blood cell or cell-derived factors, alone or in combination, to facilitate a individualized predictive parameter of responsiveness towards immunoglobin therapy.

Asphalter et al in Clin Exp Immunol 2000, 121, 506-514 reported effects of in vivo IVIG replacement therapy and high-dose IVIG (2 g/kg body weight) on NK cell subsets. They describe an assay wherein intracellular IFN-γ was measured in NK cells before and after IVIG therapy (200-400 mg/kg every 3 weeks). Ruiz. et al. in Journal of Reproductive Immunology, 31 (1996), 125-141 pertains to the immunological mechanism of IVIG to inhibit NK activity in vitro when it was added to NK cytotoxicity assays using peripheral blood lymphocytes as targets. However, in both reports these findings were not related to efficacy of IVIG therapy in individual patients

Tha-In Thanyalak et al. in BLOOD, Vol 110, No 9, 9. November 2007 report the effects of dendritic cells (DC's) matured for 18 hours in the presence of IVIG on natural killer (NK) cells. The effect of these cells on NK cell phenotype was examined by measuring the expression of Fc-gamma RIII after 5 days. INF-γ production and degranulation of NK cells was also analysed in co-cultures with IVIG-DC's after 48 hours and revealed increased expression of interferon-gamma. However, NK cells treated with IVIG without DC's showed only marginal activation. Thus, Tha-In concluded that only IVIG-DC's activate NK cell degranulation properly.

Kwak Joanne Y. H. et al. in EARLY PREGNANCY, Vol IV, pp 154-164 investigated the clinical effect of IVIG treatment in recurrent aborters with elevated NK cell levels while concomitantly receiving additional treatments. NK cytotoxicity and expression of CD16 was found to be significantly suppressed 5-7 days after IVIG infusion. There was no intention to use these findings to predict individual responsiveness toward IVIG therapy.

EP-A-1 801 234 relates to a diagnostic method to predict whether a subject is predisposed for acquiring a disease or developing an autoimmune disease by use of recombinant nucleic acid constructs. Such constructs are not used by the present application.

Park-Min Kyung-Hyun et al. in IMMUNITY, vol. 26, no. 1, January 2007, pp 67-78 describes several investigations related to the influence of IVIG on cellular responses to interferon-gamma. These investigations were mainly based on observations of Wisteria monocytogenes and on macrophages, which are not NK cells.

Meuer et al. describe a method to predict individual responsiveness towards immunoglobulin therapy by focusing on NK cell-associated and -derived factors like cytokines and enzymes and degranulation related parameters (WO2009/087219).

Beside several biochemical parameters, Meuer et al. reported that the amount of CD56-bright CD3-negative NK cells can decrease after immunoglobulin treatment in patients suffering from IMIDs. However, the predictive value for responsiveness towards immunoglobulin therapy has not been evaluated yet. The importance of B cells, functional immunologic and genetic factors was not addressed by this invention.

### BRIEF DESCRIPTION OF THE INVENTION

One object of the invention is to provide a reliable method to predict individual responsiveness towards immunoglobulin therapy, in particular to therapy with immunoglobulin G.

Another object of the invention was to provide a method to predict individual responsiveness towards immunoglobulin therapy, in particular to therapy with immunoglobulin G which method is faster than known methods.

The technical problem underlying the invention is solved by a method for determining the likelihood of response of an individual, suffering from a disease, towards immunoglobulin therapy comprising the steps of:
providing a sample containing B- and T-lymphocytes, natural killer cells, invariant T-cells and monocytes of the individual;
genotyping of at least one of the polynucleotides of an ADAMTS9-Intron; a KLHDC8A-Intron or of a flanking region of the CD14 gene, and
awarding the value of 1 for the homozygous SNP (Single Nucleotide Polymorphism) combinations, which suggests that the blood sample stems from a person which will not respond to immunoglobulin (IG) treatment, while
awarding the value of 0 for SNP not meeting that criteria, which suggests that the blood sample stems from a person which will respond to immunoglobulin treatment.

The present invention avoids the long-lasting assays as for example disclosed by Tha-In (48 hours and 5 days) performed with different cells compared to the present invention (IVIG-DC's are not used by the present invention). Furthermore Tha-In does not find an indication to use the findings for the determination of individual responsiveness towards IVIG therapy. In addition, the only comparable assay disclosed in Tha-In (NK cells treated with IVIG) showed contradictory results compared to the present invention, unfortunately, no specific conditions were published, which makes a substantial comparison impossible (page 3257, left column, lines 3-7).

The term “genotyping” is used as the skilled person understands. In particular the term means the process of determining the genotype of an individual by examining the individual's DNA sequence by using biological assays. More accurately, genotyping is the use of DNA sequences to define biological populations, by use of molecular tools Current methods of genotyping include PCR, DNA sequencing, Allele specific oligonucleotide (ASO) probes, and hybridization to DNA microarrays or beads. It is understood that genotyping is performed with a sample of an individual, i.e. ex vivo.

The homocygotes are designated in the description of the invention as AA or BB, whereas the heterocygotes are designated AB or BA.

The terms “ADAMTS9-Intron”, the “KLHDC8A-Intron” and “a flanking region of the CD14 gene” mean specific polymorphisms in the mentioned genomic segments of an individual.

“Linar Discriminant Analysis” (LDA) is a method in statistics, pattern recognition and machine learning to find a linear combination of features which characterize or separate two or more classes of objects or events. Software is commercially available under the trade name IBM® SPSS® Statistics.

In particular, the method of the invention comprises the steps of
providing a sample containing B- and T-lymphocytes, natural killer cells, invariant T-cells and monocytes of the individual;
genotyping of the ADAMTS9-Intron at Chr.3p14.1 and physical positions 64560013-64595571-64602006-64605119-64612402-64614313-64617371-64620883, which are represented in the same order by dbSNP RS ID's rs9820942, rs6780659, rs6445415, rs11721258, rs11707584, rs7652817, rs13079218, rs9819183 accessible via http://www.ncbi.nlm.nih.gov/SNP/of the National Center for Biotechnology Information, and/or of the KLHDC8A-Intron at Chr.1q32.1 and physical positions 205312280-205318524-205318854-205318983, which are represented in the same order by dbSNP RS ID's rs7549293, rs10751436, rs913723, rs913722, and/or of the CD14 flanking region at Chr.5q31.3 and physical positions 140007011-140011315-140014909-140015208, which are represented in the same order by dbSNP RS ID's rs778588, rs2563298, rs5744448, rs2569192, and awarding the value of 1 for the homozygous SNP (Single Nucleotide Polymorphism) combinations BB-AA-AA-BB-AA-BB-BB-AA of the ADAMTS9-Intron, which is represented by the homozygous SNP combination G(dbSNP RS ID rs9820942—physical position 64560013)-C(dbSNP RS ID rs6780659—physical position 64595571)-A(dbSNP RS ID rs6445415—physical position 64602006)-T(dbSNP RS ID rs11721258—physical position 64605119)-A(dbSNP RS ID rs11707584—physical position 64612402)-G(dbSNP RS ID rs7652817—physical position 64614313)-T(dbSNP RS ID rs13079218—physical position 64617371)-A(dbSNP RS ID rs9819183—physical position 64620883), AA-BB-AA-BB of the KLHDC8A-Intron, which is represented by the homozygous SNP combination C(dbSNP RS ID rs7549293—physical position 205312280)-T(dbSNP RS ID rs10751436—physical position 205318524)-A(dbSNP RS ID rs913723—physical position 205318854)-T(dbSNP RS ID rs913722—physical position 205318983), and AA-BB-BB-AA of the CD14 flanking region at said physical positions, which is represented by the homozygous SNP combination A(dbSNP RS ID rs778588—physical position 140007011)-C(dbSNP RS ID rs2563298—physical position 140011315)-C(dbSNP RS ID rs5744448—physical position 140014909)-C(dbSNP RS ID rs2569192—physical position 140015208), which suggests that the blood sample stems from a person which will not respond to IG treatment, while awarding the value of 0 for SNP not meeting that criteria, which suggests that the blood sample stems from a person which will respond to IG treatment.

The polynucleotides for genotyping according to the present invention have been derived from http://www.affymetrix.com/analysis/netaffx/index.affx.

More detailed information about said SNP's are presented in table 1 and can be retrieved from the SNP-database (http://www.ncbi.nlm.nih.gov/SNP/) of the National Center for Biotechnology Information.

In a further embodiment of the invention the genotyping status is complemented with parameters by determination of at least one of the amount of cytokines released from or their expressed genes on cells, wherein cytokines are selected from the group of Interferon-gamma (IFN-γ), Interleukin-8 (CXCL8), C-X-C motif chemokine 10 (CXCL10), chemokine C-C motif ligand 8 (CCL8), chemokine C-C motif ligand 20 (CCL20), Interleukin-10 (IL-10) and Stem cell factor (SCF).

In still a further embodiment of the invention the genotyping status is complemented with parameters by determination of the amount at least one of the proteins CD32b, CD16, IL-6R (Interleukin-6 receptor) and ICAM-1 (Inter Cellular Adhesion Molecule 1) released from and/or or their expressed genes on cells.

In yet a further embodiment the method of the invention the release of said proteins and the expression of their genes is determined after ex vivo exposure of samples with immunoglobulin, in particular IgG, IgM, IgA or a combination thereof.

In particular, the genotyping, the protein release and the gene expression is determined in whole blood, blood fractions, cell fractions or plasma.

According to the invention, in particular, a sample is incubated in presence of a stimulant in at least one assay in presence of immunoglobulins and in at least one assay in absence of immunoglobulins as control and wherein the stimulant is selected from the group consisting of lipopolysaccharides (LPS), phorbol-12-myristate-13 acetate PMA)/ionomycin, monoclonal antibodies binding to receptors on leukocytes or combinations thereof.

In particular, the amount of immunoglobulins used in assays is from about 0.01 to about 100 mg/ml in particular from about 1 to about 50 mg/ml.

Typically, the method of the invention may be performed before and/or during the treatment of a patient with immunoglobulin.

In a further embodiment of the invention the genotyping status of the ADAMTS9-Intron is complemented by the parameter “IG induced (netto) CCL20 release” and the LDA-Score (Linear Discriminant Analysis) determined by incorporation of the value for genotyping status and the value of protein amount given in [pg/ml] into the formula:

LDA−LDA-Score(2P5)=12,6661481683*(ADAMTS9 Genotype)−0,0018215212*(IG induced(netto)CCL20 release)−5,2193484355,

wherein a LDA-Score(2P5)≦0.0 indicates responders while a LDA-Score(2P5)>0.0 indicates non-responders.

In still a further embodiment of the invention the genotyping status of the ADAMTS9-Intron is complemented by the parameter “IG induced (netto) CCL8 release” and the LDA-Score determined by incorporation of the value for genotyping status and the value of protein amount given in [pg/ml] into the formula:

LDA-Score(2P4)=5,1547784757*(ADAMTS9 Genotype)+0,0006613541*(IG induced (netto) CCL8 release)−2,5483009377,

wherein a LDA-Score(2P4)≦0.0 indicates responders while a LDA-Score(2P4)>0.0 indicates non-responders.

In yet a further embodiment of the invention the genotyping status of the ADAMTS9-Intron is complemented by the parameter “IG induced (netto) IL-10 release” and the LDA-Score determined by incorporation of the value for genotyping status and the value of protein amount given in [pg/ml] into the formula:

LDA-Score(2P3)=5,1710817023*(ADAMTS9 Genotype)−0,0526409406*(IG induced (netto) IL-10 release)−2,5189275627,

wherein a LDA-Score(2P3)≦0.0 indicates responders while a LDA-Score(2P3)>0.0 indicates non-responders.

In a further embodiment of the invention the genotyping status of the ADAMTS9-Intron is complemented by the parameter “IG/LPS induced (netto) IFN-γ Genex” and the LDA-Score determined by incorporation of the value for genotyping status and the value of transcript numbers given in [transcripts/μl] into the formula:

LDA-Score(2P2)=5,1584234532*(ADAMTS9 Genotype)+0,0009843151*(IG/LPS induced (netto) IFN-γ Genex)−2,5250980052,

wherein a LDA-Score(2P2)≦0.0 indicates responders while a LDA-Score(2P2)>0.0 indicates non-responders.

In a further embodiment of the invention the genotyping status of the ADAMTS9-Intron is complemented by the parameter “IG induced (netto) IFN-γ Genex” and the LDA-Score determined by incorporation of the value for genotyping status and the value of transcript numbers given in [transcripts/μl] into the formula:

LDA-Score(2P1)=5,173156752*(ADAMTS9 Genotype)+0,0010883751*(IG induced (netto) IFN-γ Genex)−2,5111538246,

wherein a LDA-Score(2P1)≦0.0 indicates responders while a LDA-Score(2P1)>0.0 indicates non-responders.

In a further embodiment of the invention the genotyping status of the ADAMTS9-Intron is complemented by the parameters “IG induced (netto) CCL20 release” and “IG induced (netto) CCL8 release” and the LDA-Score determined by incorporation of the value for genotyping status and the values of transcript numbers and protein amount given in [transcripts/μl] and [pg/ml] into the formula:

LDA-Score(3P2)=28,427707664*(ADAMTS9 Genotype)−0,0046972337*(IG induced (netto) CCL20 release)+0,0129144727*(IG induced (netto) CCL8 release)−12,7163079623,

wherein a LDA-Score(3P2)≦0.0 indicates responders while a LDA-Score(3P2)>0.0 indicates non-responders.

In a further embodiment of the invention the genotyping status of the ADAMTS9-Intron is complemented by the parameters “IG induced (netto) CCL20 release”, “IG induced (netto) CCL8 release”, and “IG/LPS induced (netto) IFN-γ Genex” and the LDA-Score determined by incorporation of the value for genotyping status and the values of transcript numbers and protein amount given in [transcripts/μl] and [pg/ml] into the formula:

LDA-Score(4P1)=31,5741470438*(ADAMTS9 Genotype)−0,0052245002*(IG induced (netto) CCL20 release)+0,0166330872*(IG induced (netto) CCL8 release)−0,0109678784*(IG/LPS induced (netto) IFN-γ Genex)−13,8885092449,

wherein a LDA-Score(4P1)≦0.0 indicates responders while a LDA-Score(4P1)>0.0 indicates non-responders.

In a further embodiment of the invention the genotyping status of the ADAMTS9-Intron is complemented by the parameters “IG induced (netto) CCL20 release”, “IG induced (netto) CCL8 release”, “IG induced (netto) CXCL8 release” and “IG/LPS induced (netto) IFN-γ Genex” and the LDA-Score determined by incorporation of the value for genotyping status and the values of transcript numbers and protein amount given in [transcripts/μl] and [pg/ml] into the formula:

LDA-Score(5P1)=107,2468831*(ADAMTS9 Genotype)−0.038780771*(IG/LPS induced (netto) IFN-γ Genex)−0.017866668*(IG induced (netto) CCL20 release)+0.044172208*(IG induced (netto) CCL8 release)+0.002477736*(IG induced (netto) CXCL8 release)−47.9651381,

wherein a LDA-Score(5P1)≦0.0 indicates responders while a LDA-Score(5P1)>0.0 indicates non-responders.

In a further embodiment of the invention the genotyping status of the ADAMTS9-Intron is complemented by the parameters “IG induced (netto) CCL20 release”, “IG induced (netto) CCL8 release”, “IG induced (netto) SCF release” and “IG/LPS induced (netto) IFN-γ Genex” and the LDA-Score determined by incorporation of the value for genotyping status and the values of transcript numbers and protein amount given in [transcripts/μl] and [pg/ml] into the formula:

LDA-Score(5P2)=89.56250541*(ADAMTS9 Genotype)−0.128146913*(IG/LPS induced (netto) IFN-γ Genex)−0.015495947*(IG induced (netto) CCL20 release)+0.058499044*(IG induced (netto) CCL8 release)+0.008472595*(IG induced (netto) SCF release)−43.33685048,

wherein a LDA-Score(5P2)≦0.0 indicates responders while a LDA-Score(5P2)>0.0 indicates non-responders.

In a further embodiment of the invention the genotyping status of the ADAMTS9-Intron is complemented by the parameters “IG induced (netto) CCL20 release”, “IG induced (netto) CCL8 release”, “IG induced (netto) CXCL10 release”, “IG induced (netto) IL-10 release”, “IG induced (netto) CXCL8 release”, “IG induced (netto) ICAM1 Genex”, “IG/LPS induced (netto) IFN-γ Genex” and “IG induced (netto) CXCL8 Genex” and the LDA-Score determined by incorporation of the value for genotyping status and the values of transcript numbers and protein amount given in [transcripts/μl] and [pg/ml] into the formula:

LDA-Score(9P)=108,5705785*(ADAMTS9 Genotype)−0.065661811*(IG induced (netto) ICAM1 Genex)−0.14179279*(IG induced (netto) IFN-γ Genex)−0.00521369*(IG induced (netto) CXCL8 Genex)−0.017983675*(IG induced (netto) CCL20 release)+0.018722767*(IG induced (netto) CCL8 release)+0.001625748*(IG induced (netto) CXCL10 release)+0.425763386*(IG induced (netto) IL-10 release)+0.004389251*(IG induced (netto) CXCL8 release)−48.34366669,

wherein a LDA-Score(9P)≦0.0 indicates responders while a LDA-Score(9P)>0.0 indicates non-responders.

In a further embodiment of the invention the genotyping status of the KLHDC8A-Intron is complemented by the parameter “IG induced (netto) IL-6R release” and the LDA-Score determined by incorporation of the value for genotyping status and the value of protein amount given in [pg/ml] into the formula:

LDA-Score(2P6)=5,6030684062*(KLHDC8A Genotype)+0,5886545649*(IG induced (netto) IL-6R release)−2,0540283735,

wherein a LDA-Score(2P6)≦0.0 indicates responders while a LDA-Score(2P6)>0.0 indicates non-responders.

In a further embodiment of the invention the genotyping status of the KLHDC8A-Intron is complemented by the parameter “IG induced (netto) ICAM1 release” and the LDA-Score determined by incorporation of the value for genotyping status and the value of protein amount given in [pg/ml] into the formula:

LDA-Score(2P7)=6,4011642693*(KLHDC8A Genotype)+0,1385143298*(IG induced (netto) ICAM1 release)−2,870032934,

wherein a LDA-Score(2P7)≦−1.0 indicates responders while a LDA-Score(2P7)>−1.0 indicates non-responders.

In a further embodiment of the invention the genotyping status of the KLHDC8A-Intron is complemented by the parameter “IG induced (netto) CD32b Genex” and the LDA-Score determined by incorporation of the value for genotyping status and the values of transcript numbers given in [transcripts/μl] into the formula:

LDA-Score(2P8)=5,7296431439*(KLHDC8A Genotype)−0,3050588266*(IG induced (netto) CD32b Genex)−2,8435304986,

wherein a LDA-Score(2P8)≦−1.0 indicates responders while a LDA-Score(2P8)>−1.0 indicates non-responders.

In a further embodiment of the invention the genotyping status of the KLHDC8A-Intron is complemented by the parameters “IG/LPS induced (netto) CD16 Genex” and “IG induced (netto) ICAM-1 release” and the LDA-Score determined by incorporation of the value for genotyping status and the values of transcript numbers and protein amount given in [transcripts/μl] and [pg/ml] into the formula:

LDA-Score(3P1)=6,207662683*(KLHDC8A Genotype)−0.007323378*(IG/LPS induced (netto) CD16 Genex)+0.219033761*(IG induced (netto) ICAM-1 release)−5.456170752,

wherein a LDA-Score(3P1)≦−1.0 indicates responders while a LDA-Score(3P1)>−1.0 indicates non-responders.

In a further embodiment of the invention the genotyping status of the KLHDC8A-Intron is complemented by the parameters “IG/LPS induced (netto) CD32b Genex”, “IG induced (netto) IL-6R release” and “IG induced (netto) ICAM1 release” and the LDA-Score determined by incorporation of the value for genotyping status and the values of transcript numbers and protein amount given in [transcripts/μl] and [pg/ml] into the formula:

LDA-Score(4P2)=7,0639539622*(KLHDC8A Genotype)−0,2539770554*(IG/LPS induced (netto) CD32b Genex)+0,4613873178*(IG induced (netto) IL-6R release)+0,111066766*(IG induced (netto) ICAM1 release)−3,3328149764,

wherein a LDA-Score(4P2)≦0.0 indicates responders while a LDA-Score(3P1)>0.0 indicates non-responders.

In a further embodiment of the invention the genotyping status of the KLHDC8A-Intron is complemented by the parameters “IG/LPS induced (netto) CD16 Genex”, “IG/LPS induced (netto) CD32b Genex”, “IG induced (netto) ICAM-1 release” and “IG induced (netto) IL-6R release” and the LDA-Score determined by incorporation of the value for genotyping status and the values of transcript numbers and protein amount given in [transcripts/μl] and [pg/ml] into the formula:

LDA-Score(5P3)=11.44098342*(KLHDC8A Genotype)−0.045599133*(IG/LPS induced (netto) CD16 Genex)−0.535989358*(IG/LPS induced (netto) CD32b Genex)+0.225465018*(IG induced (netto) ICAM-1 release)+3.14495298*(IG induced (netto) IL-6R release)−3.9398568,

wherein a LDA-Score(5P3)≦0.0 indicates responders while a LDA-Score(5P3)>0.0 indicates non-responders.

In a further embodiment of the invention the genotyping status of the KLHDC8A-Intron is complemented by the parameters “IG induced (netto) CD32b Genex”, “IG induced (netto) ICAM-1 Genex”, “IG/LPS induced (netto) CD32b Genex” and “IG induced (netto) IL-6R release” and the LDA-Score determined by incorporation of the value for genotyping status and the values of transcript numbers and protein amount given in [transcripts/μl] and [pg/ml] into the formula:

LDA-Score(5P4)=9.476844721*(KLHDC8A Genotype)−0.361944446*(IG induced (netto) CD32b Genex)+0.008332887*(IG induced (netto) ICAM-1 Genex)−0.939416614*(IG/LPS induced (netto) CD32b Genex)+0,951418988*(IG induced (netto) IL-6R release)−4.232325519,

wherein a LDA-Score(5P4)≦0.0 indicates responders while a LDA-Score(5P4)>0.0 indicates non-responders.

In still a further embodiment of the invention the indication of responder or non-responder is confirmed by at least one additional method.

In a further embodiment of the invention any immunoglobulin product suitable for in vivo use is concerned such as those applied intravenously, subcutaneously, intramuscularly, ocularly, intrathecially, orally, topically or inhalably.

In a further embodiment of the invention the disease is selected from the group consisting of inflammatory mediated immune diseases, autoimmune diseases, allergies, graft-versus-host reactions and prevention of transplant rejection; any kind of multiple sclerosis or any other demyelinating neurological disease; or re-lapsing-remitting multiple sclerosis.

In a further embodiment of the invention is used for permitting to predict the probability of a relapse of a MS patient and/or the rate of progression of the disease in terms of disability and or functioning of the patient as measured by clinical scales such as, but not limited to, the expanded disability status scale (EDSS), in particular lupus erythematosus, rheumatoid arthritis or intestinal/bowel diseases such as Crohn's disease, myositis or recurrent abortion.

Subject matter of the invention is also the use of the method of the invention for facilitating the approval or recommendation of immunoglobulins by health authorities for the treatment of any kind of multiple sclerosis or any other demyelinating disease or Lupus erythematosus, rheumatoid arthritis or intestinal/bowel diseases such as Crohn's disease, myositis or recurrent abortion.

### BRIEF DESCRIPTION OF THE FIGURES

FIG. 1—FIG. 20 show in diagrams LDA-scores.

### DETAILED DESCRIPTION OF THE INVENTION

The technical problem underlying the invention is solved by a method predicting individual responsiveness towards immunoglobulin therapy, wherein the homocygocity of certain regions of ADAMTS9-Intron (intron of A disintegrin and metalloproteinase with thrombospondin motifs 9; at Chr.3p14.1 and physical positions 64560013, 64595571, 64602006, 64605119, 64612402, 64614313, 64617371, 64620883, respectively in the same order dbSNP RS ID's rs9820942, rs6780659, rs6445415, rs11721258, rs11707584, rs7652817, rs13079218, rs9819183) and KLHDC8A-Intron (intron of kelch domain containing 8A; Chr. 1q32.1 and physical positions 205312280-205318524-205318854-205318983, respectively in the same order dbSNP RS ID's rs7549293, rs10751436, rs913723, rs913722SNP's) or CD14 flanking region (at Chr. 5q31.3 (SNP Chromosom 5, Cytoband q31.3, physical positions 140007011, 140011315, 140014909, 140015208, respectively in the same order dbSNP RS ID's rs778588, rs2563298, rs5744448, rs2569192)), are determined as well as the amount of cytokines released from or their expressed genes on cells is determined. The term cytokine is to be understood as including its subgroup chemokines. The cytokines, respectively their expressed genes, are selected from the group of Interferon-gamma (IFN-γ), Interleukin-8 (IL-8 or CXCL8), C—X-C motif chemokine 10 (CXCL10 or Interferon gamma-induced protein 10 kDa (IP-10)), chemokine C-C motif ligand 8 (CCL8), chemokine C-C motif ligand 20 (CCL20), Interleukin-10 (IL-10) and Stem cell factor (SCF) and are used for prediction of IgG responsiveness alone or in combination with others. Also of interest is the release and/or gen-expression of CD32b, CD16 (FcR-γ III), CD 19, CD20, CD56, IL-6R (Interleukin-6 receptor) and ICAM-1 (Inter Cellular Adhesion Molecule 1).

Cited cellular-biochemical parameters were studied in whole blood and derived from plasma and or leukocytes of patients suffering from relapsing-remitting multiple sclerosis by analyzing and comparing blood samples, drawn before and after administration of immunoglobulin (regular administration of 0.4 g IVIG/kg bodyweight). The study initially incorporated 33 individuals of which 6 were later excluded due to displaying obvious inflammatory activity or dropout. Analytical results of remaining 27 patients, 15 were found to be responders and 12 non-responders according to study-design, were thus used for determination of relevant parameters.

While each of cited parameter revealed a certain predictive value after ROC-analysis (Receiver Operating Characteristic) on its own it was found, that a combination of one or more parameters based on cytokine (IFN-γ, CXCL8, CXCL10, IL-10 and SCF) release and/or gen-expression and genotyping of ADAMTS9-Intron or based on release and/or gen-expression of CD16, CD32b, IL-6R and ICAM-1 and genotyping of KLHDC8A-Intron was much more predictive as a strong correlation exists between responders to immunoglobulin treatment and said parameters with respect to prevention of relapses or extension of remission. It is also possible to combine the results of one of the genotyping assays with one or two of the other genotyping assays in order to identify possible false positive/negative results or to confirm the assessment of responders/non-responders. The confirmation of a responder determined by KLHDC8A-Intron genotyping by genotyping of the ADAMTS9-Intron can be seen as a non-limiting example for such a combination. It may be useful to use the genotyping results of the introns KLHDC8A and CD14, or ADAMTS9 and CD14, or even KLHDC8A and ADAMTS9 and CD14.

Furthermore, these parameters can further be combined with other parameters increasing the predictability of immunoglobulin therapy response, e.g. NK cell degranulation parameters (granzyme B, perforin, or CD107a) and functional NK cell killing activity or combinations thereof. All of these parameters (cytokine release, cytokine gen-expression, NK cell degranulation parameters and functional NK cell killing activity) are generated in short-time ex vivo cultures of whole blood samples or plasma exposed to immunoglobulin (with or without LPS (lipopolysaccharide) stimulation).

### Reagents and Assays

Reagents, assays and assessment of results are well known to persons skilled in the art. Many assays were described in detail by Jacobi et. al. in Clinical Immunology (2009) 133, 393-401.

General preparation of whole blood cultures was performed by admixture of heparinized venous whole blood to the same volume of a stock solution containing 20 mg/ml IgG in culture medium (RPMI—1640, 10% FCS, L-glutamine, penicillin, streptomycin, 50 μM β-mercaptoethanol) with. This mixture was incubated at 37° C. for 3 h, when qRT-PCR, FACS and NK cell killing-assays were to be performed, and for 24 h, when ELISAs were to be performed. Such assays are called “IG induced” throughout this application. Some assays were performed with lipopoliysaccaride (LPS, purchased from Sigma, St. Luis, USA) at 100 ng/ml final concentration in the mixture of whole blood, IgG and culture medium for stimulation. Such assays are called “IG/LPS induced” throughout this application. The same assays were also performed with maltose 10 mg/ml final concentration instead of IgG for comparison with IgG incubated assays. The difference between IG or IG/LPS incubated minus maltose or maltose/LPS (control samples as IG is missing) incubated assay results are denominated “(netto)” throughout this application.

Genotyping was performed with whole blood on the GeneChip® Human Mapping 6.0 Array from Affymetrix according to the protocol of the manufacturer. Homozygous SNP (Single Nucleotide Polymorphism) combinations at relevant positions were incorporated in data analysis with a value of 1 while SNP not meeting that criteria received the value 0. In particular, the homozygous sequence of AA-BB-BB-AA of the CD14 flanking region (CHR.5q31.3), which is represented by the homozygous SNP combination A(dbSNP RS ID rs778588—physical position 140007011)-C(dbSNP RS ID rs2563298—physical position 140011315)-C(dbSNP RS ID rs5744448—physical position 140014909)-C(dbSNP RS ID rs2569192—physical position 140015208) and the homozygous sequence of AA-BB-AA-BB of the KLHDC8A-Intron (Chr. 1q32.1), which is represented by the homozygous SNP combination C(dbSNP RS ID rs7549293—physical position 205312280)-T(dbSNP RS ID rs10751436—physical position 205318524)-A(dbSNP RS ID rs913723—physical position 205318854)-T(dbSNP RS ID rs913722—physical position 205318983), as well as the homozygous sequence of BB-AA-AA-BB-AA-BB-BB-AA of the ADAMTS9-Intron at Chr. 3p14.1, which is represented by the homozygous SNP combination G(dbSNP RS ID rs9820942—physical position 64560013)-C(dbSNP RS ID rs6780659—physical position 64595571)-A(dbSNP RS ID rs6445415—physical position 64602006)-T(dbSNP RS ID rs11721258—physical position 64605119)-A(dbSNP RS ID rs11707584—physical position 64612402)-G(dbSNP RS ID rs7652817—physical position 64614313)-T(dbSNP RS ID rs13079218—physical position 64617371)-A(dbSNP RS ID rs9819183—physical position 64620883), were given the value of 1, while all other sequences were given the value of 0.

Gen-expression assays were performed with whole blood by qRT-PCR. Cells were resuspended, after red cell lysis, in 400 μl MagNApure Lysis buffer (Roche Applied Science, Mannheim, Germany) and lysates were stored at −80° C. until analysis. An automated sample preparation system (MagNA-Pure, Roche Applied Science, Mannheim, Germany) was used for mRNA isolation according to the manufactures protocol. The elution volume was set to 50 μl. An aliquot of 8.2 μl RNA was reverse transcribed using a first strand cDNA synthesis kit (Roche Applied Science, Mannheim, Germany) and oligo-(dT) as primer using the manufacturer's protocol in a thermocycler. After termination of the cDNA synthesis, the reaction mix was diluted to a final volume of 500 μl and stored at −20° C. until polymerase chain reaction (PCR) analysis. Parameter specific primer sets optimized for the LightCycler (Roche Applied Science, Mannheim, Germany) were developed and purchased from SEARCH-LC GmbH (Heidelberg, Germany). The PCR was performed with the LightCycler FastStart DNA Sybr Green I kit (Roche Applied Science, Mannheim, Germany) according to the protocol provided in the parameter specific kits. RNA input was normalized by the average expression of the two housekeeping genes β-actin and Cyclophilin B. The data was used as adjusted transcripts per μl cDNA.

Analysis of released proteins was performed by ELISA in supernatants of whole blood cultures by using commercially available kits (Diaclone, Pelikine and Luminex) according to the manufacturer's protocols. Whole blood cultures were sedimented by centrifugation after 24 h of incubation at 37° C. and supernatants were kept at −80° C. until assay. The data was used as pg/ml protein concentration.

Any other change of cellular marker associated with degranulation can be utilized to detect and quantify the degranulation efficacy and status induced by IgG exposure. Representatives of such NK cell granulae (lytic lysosomes) compounds are the proteins perforin and granzymes (the latter proteases, more specifically granzyme B), which can be quantified by for instance antigen detection systems like ELISA or direct enzymatic tests (for enzymes and proteases). The increased expression of CD107a is a typical indicator of NK degranulation. Using lipopolysaccharide (bacteria derived) in a whole blood assay system and thereby mimicking a patho-physiological situation, both IFN-gamma and CXCL10 are up-regulated on mRNA transcript (number) and protein (release) level. It was found that in the presence of added immunoglobulin (upon stimulation with LPS), the increase of CXCL10 was reduced by immunoglobulins as compared to the control without immunoglobulins, whereas no relative reduction was observed for IFN-γ.

In general, the monitored signal is measured by methods known to the expert, like specific detection by using a labeled antibody, fragment or affinity ligand in flow cytometry (like FACS, fluorescent-activated cell sorting).

Moreover, these parameters can be combined with assays on single nucleotide polymorphisms (SNP) like Interleukin-2 receptor (IL-2R), interleukin-7 receptor (IL7-R), and CD58 or with assays for genotyping indicating the patients different genetic background.

All those analytical results were correlated with the outcome of the study, differentiation between patients responding to IgG-treatment (“responders”) and non responding patients (“non-responders”), and subjected to Linear Discriminant Analysis (LDA) for determination of most relevant parameters for prediction of a person's susceptibility to IgG treatment. After identification of useful parameters several LDA-scores with high predictive value were established, as displayed by the following examples. Analytical results were introduced in respective formula for LDA-Sore calculation in values and dimensions as indicated in the general assay description. Many examples were established by a random subset of responders and non-responders (indicated in the figures as “Learning Set”) of approximately the same size while the complement subset (“Validation Set”) was tested with the established formula. And both subsets were also of similar sizes.

General description of abbreviations used in the examples, as already discussed during general description of assays:

“IG” indicates incubation of probe with immunoglobulin
“IG/LPS” indicates incubation of probe with immunoglobulin and LPS stimulation
“X Genex” indicates gen-expression of X
“Y release” indicates release/degranulation of protein Y
“(netto)” indicates incubation for 3 h when used in combination with “Genex” respectively incubation for 24 h when used in combination with “release” and sub-traction of the analytical value of control samples (maltose or maltose/LPS) from immunoglobulin incubated samples (IG or IG/LPS).

### Example 1

### LDA-Score (9 Parameters)

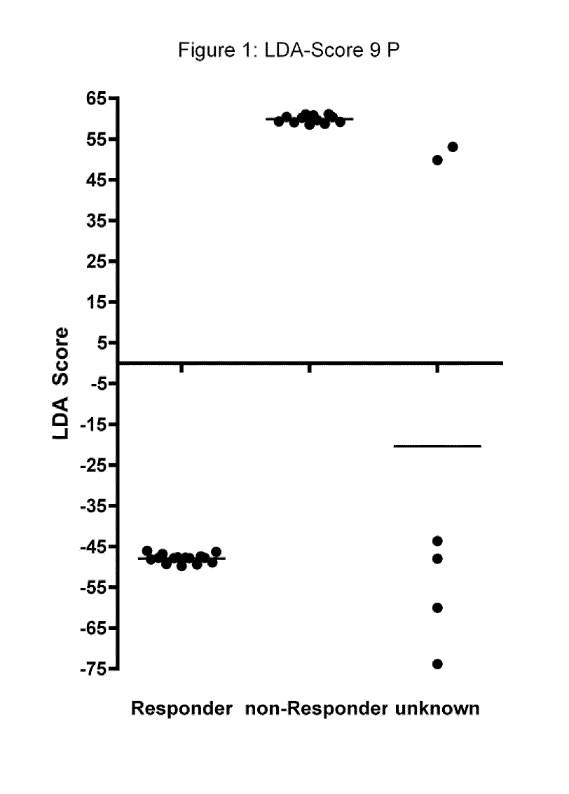

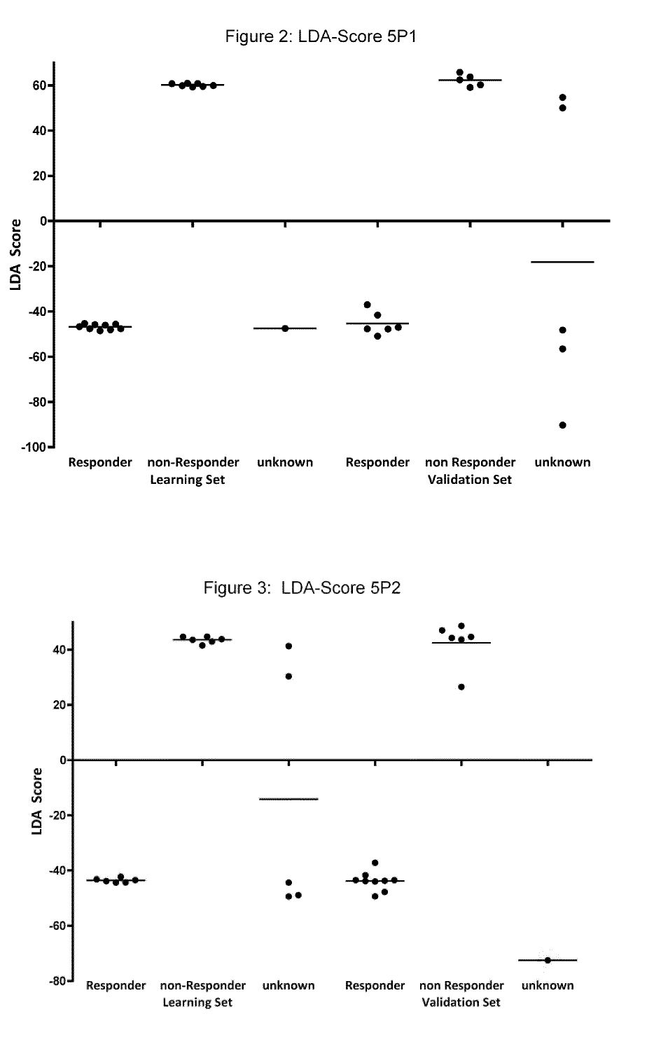

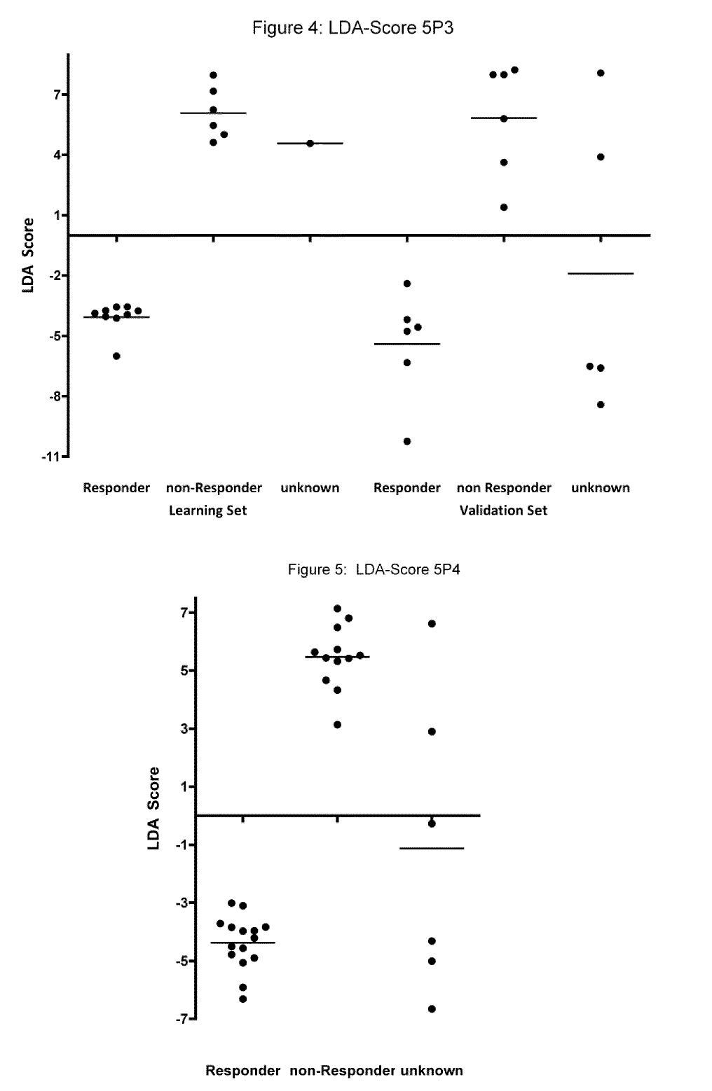

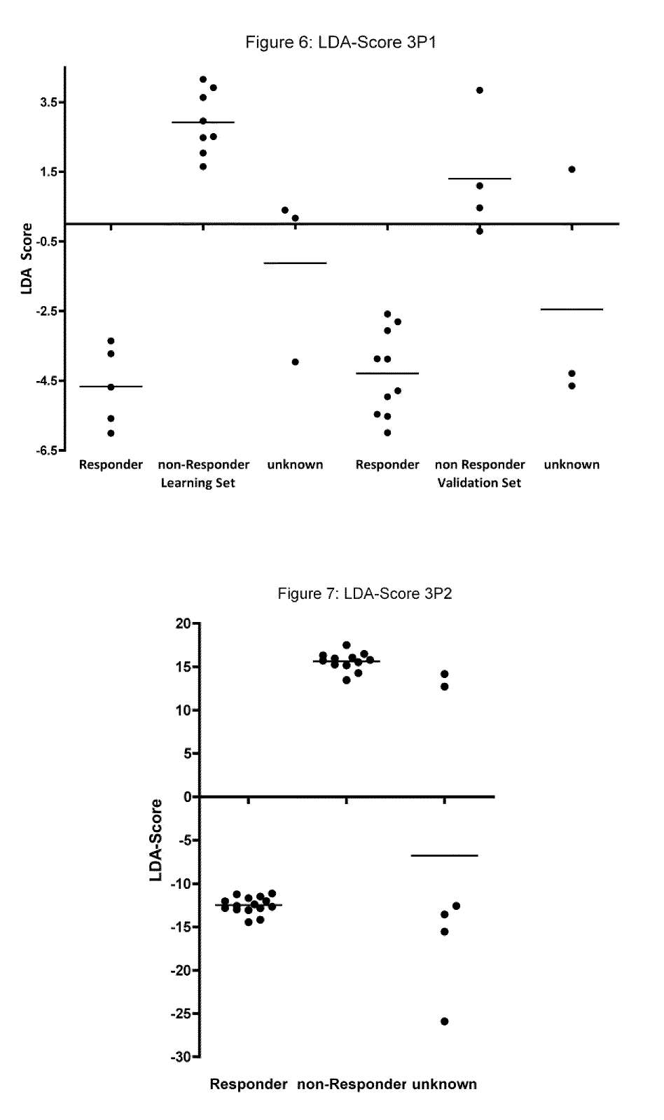

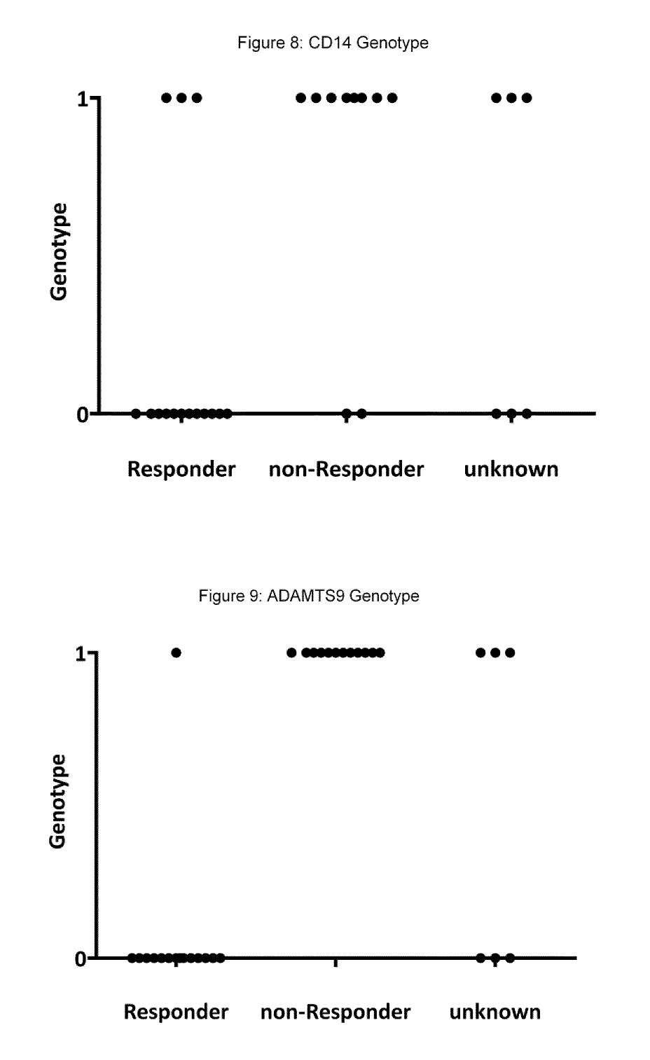

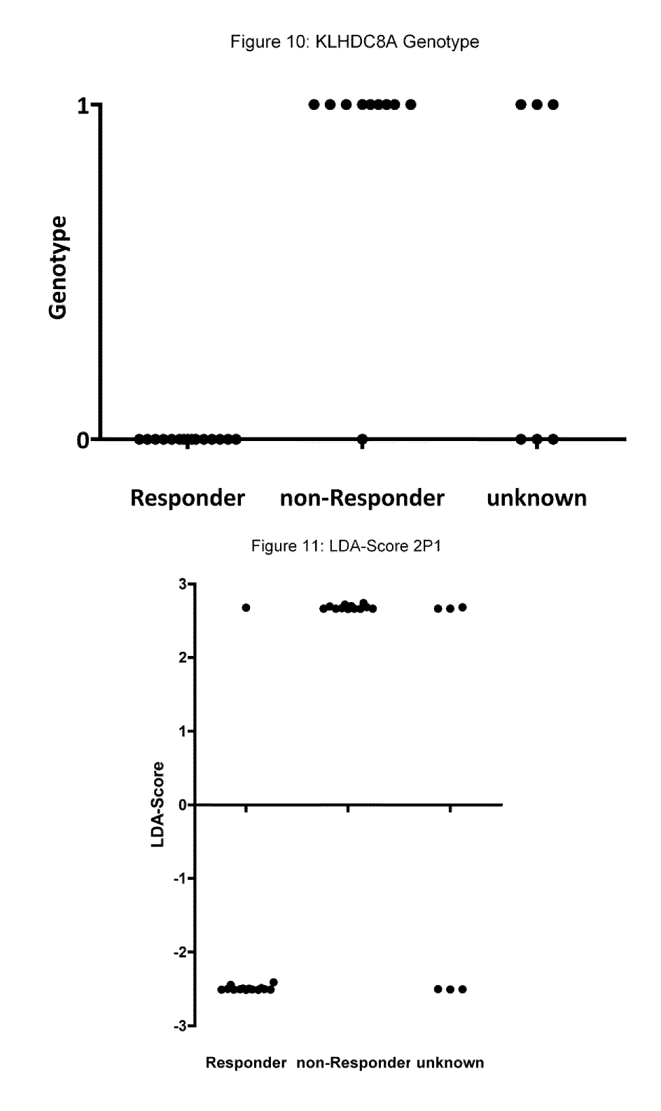

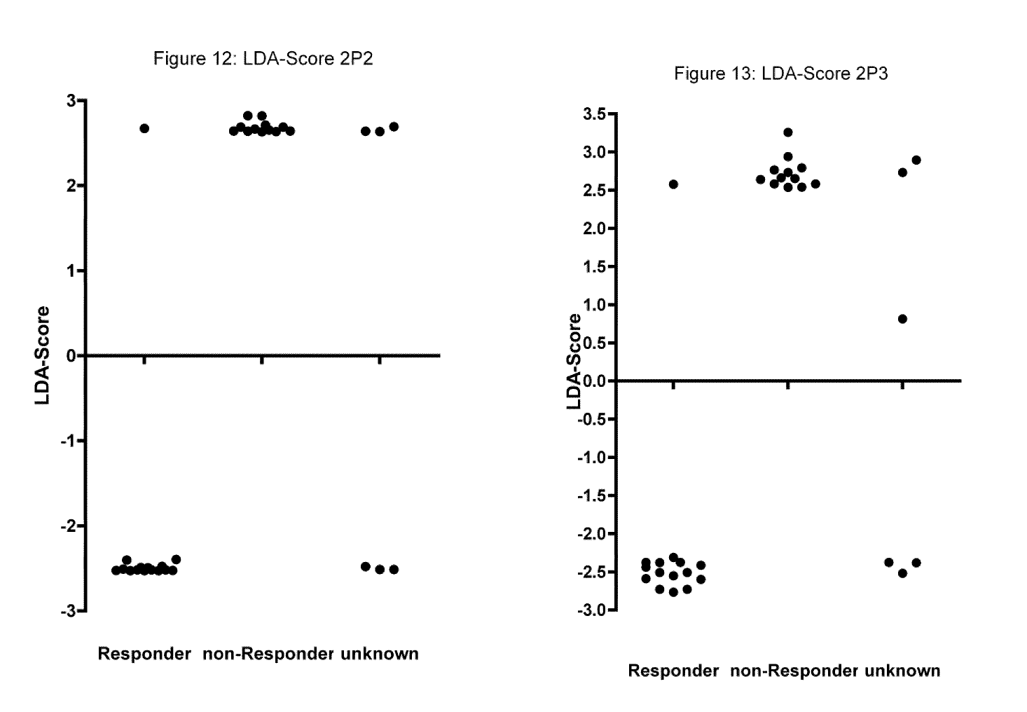

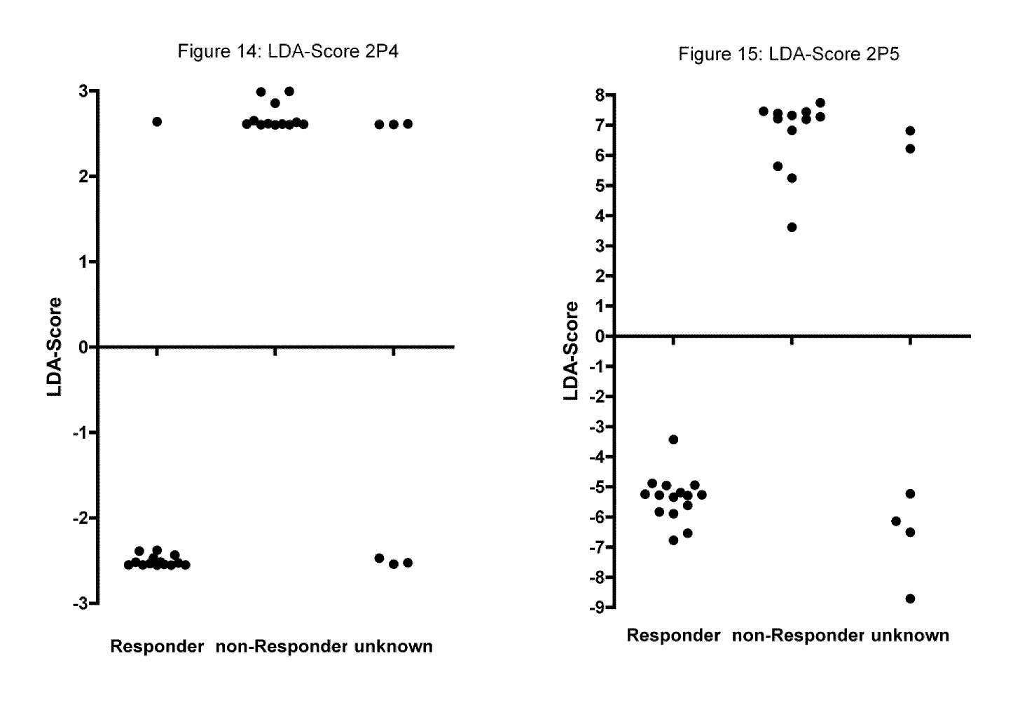

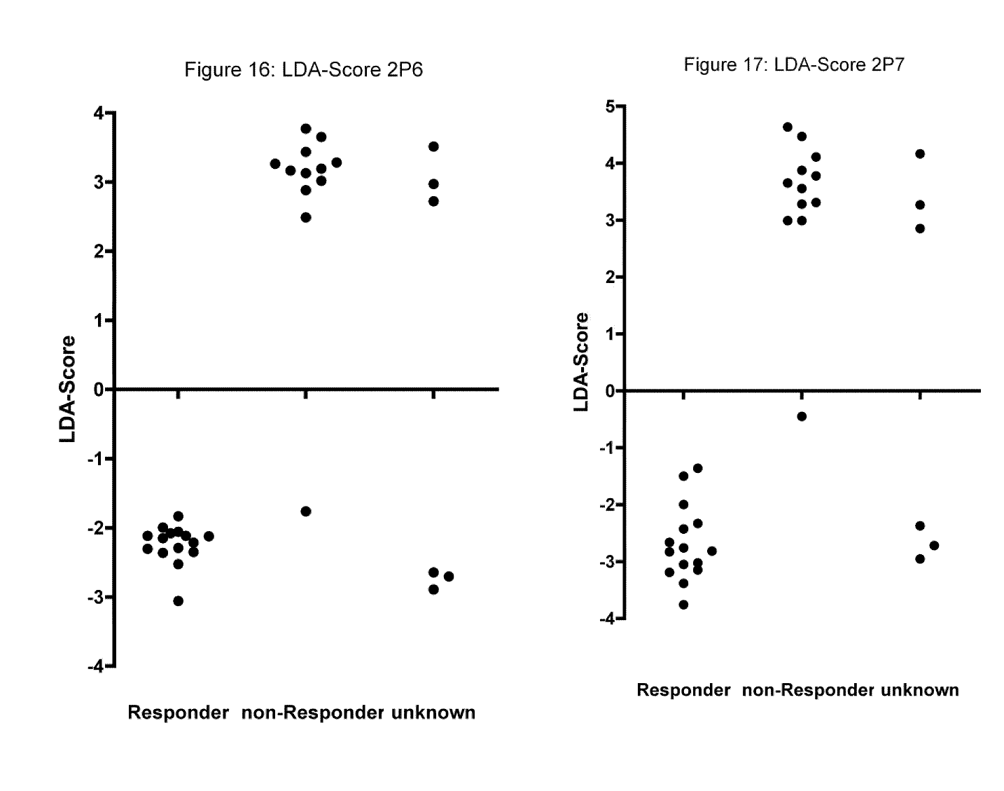

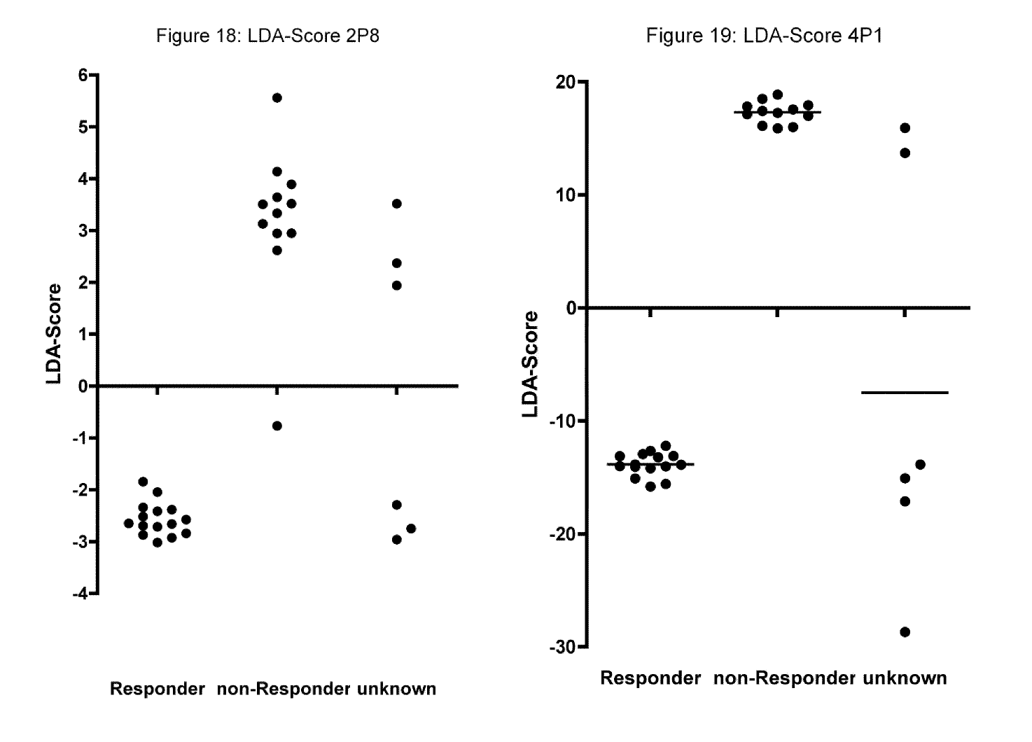

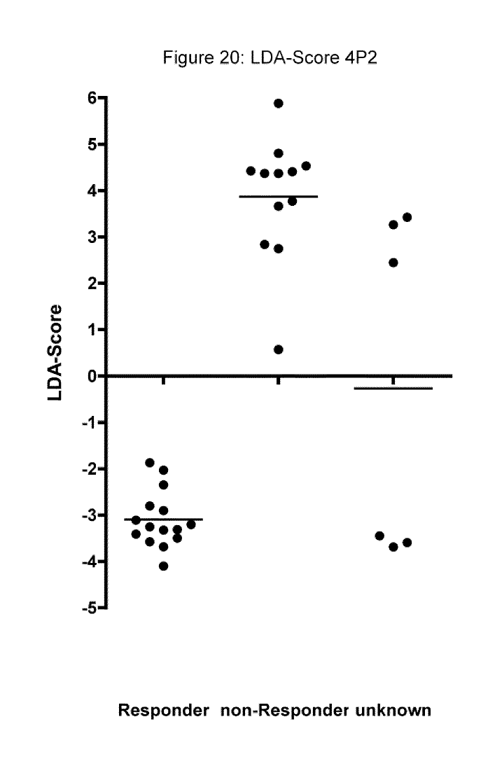

LDA-Score(9P)=108,5705785*(ADAMTS9 Genotype)−0.065661811*(IG induced (netto) ICAM1 Genex)−0.14179279*(IG induced (netto) IFN-γ Genex)−0.00521369*(IG induced (netto) CXCL8 Genex)−0.017983675*(IG induced (netto) CCL20 release)+0.018722767*(IG induced (netto) CCL8 release)+0.001625748*(IG induced (netto) CXCL10 release)+0.425763386*(IG induced (netto) IL-10 release)+0.004389251*(IG induced (netto) CXCL8 release)−48.34366669
A LDA-Score(9P)≦0.0 indicates responders while a LDA-Score(9P)>0.0 indicates non-responders as depicted in FIG. 1.

### Example 2

### LDA-Score (5P1)

LDA-Score(5P1)=107,2468831*(ADAMTS9 Genotype)−0.038780771*(IG/LPS induced (netto) IFN-γ Genex)−0.017866668*(IG induced (netto) CCL20 release)+0.044172208*(IG induced (netto) CCL8 release)+0.002477736*(IG induced (netto) CXCL8 release)−47.96513813
A LDA-Score(5P1)≦0.0 indicates responders while a LDA-Score(5P1)>0.0 indicates non-responders as depicted in FIG. 2.

### Example 3

### LDA-Score (5P2)

LDA-Score(5P2)=89.56250541*(ADAMTS9 Genotype)−0.128146913*(IG/LPS induced (netto) IFN-γ Genex)−0.015495947*(IG induced (netto) CCL20 release)+0.058499044*(IG induced (netto) CCL8 release)+0.008472595*(IG induced (netto) SCF release)−43.33685048
A LDA-Score(5P2)≦0.0 indicates responders while a LDA-Score(5P2)>0.0 indicates non-responders as depicted in FIG. 3.

### Example 4

### LDA-Score (5P3)

LDA-Score(5P3)=11.44098342*(KLHDC8A Genotype)−0.045599133*(IG/LPS induced (netto) CD16 Genex)−0.535989358*(IG/LPS induced (netto) CD32b Genex)+0.225465018*(IG induced (netto) ICAM-1 release)+3.14495298*(IG induced (netto) IL-6R release)−3.9398568
A LDA-Score(5P3)≦0.0 indicates responders while a LDA-Score(5P3)>0.0 indicates non-responders as depicted in FIG. 4.

### Example 5

### LDA-Score (5P4)

LDA-Score(5P4)=9.476844721*(KLHDC8A Genotype)−0.361944446*(IG induced (netto) CD32b Genex)+0.008332887*(IG induced (netto) ICAM-1 Genex)−0.939416614*(IG/LPS induced (netto) CD32b Genex)+0,951418988*(IG induced (netto) IL-6R release)−4.232325519
A LDA-Score(5P4)≦0.0 indicates responders while a LDA-Score(5P4)>0.0 indicates non-responders as depicted in FIG. 5.

### Example 6

### LDA-Score (3P1)

LDA-Score(3P1)=6,207662683*(KLHDC8A Genotype)−0.007323378*(IG/LPS induced (netto) CD16 Genex)+0.219033761*(IG induced (netto) ICAM-1 release)−5.456170752
A LDA-Score(3P1)≦−1.0 indicates responders while a LDA-Score(3P1)>−1.0 indicates non-responders as depicted in FIG. 6.

### Example 7

### LDA-Score (3P2)

LDA-Score(3P2)=28,427707664*(ADAMTS9 Genotype)−0,0046972337*(IG induced (netto) CCL20 release)+0,0129144727*(IG induced (netto) CCL8 release)−12,7163079623.
A LDA-Score(3P2)≦0.0 indicates responders while a LDA-Score(3P2)>0.0 indicates non-responders as depicted in FIG. 7.

### Example 8

### CD14-Genotype

The homozygous sequence of AA-BB-BB-AA for the SNP's rs7549293, rs10751436, rs913723, rs913722 of the CD14 flanking region at CHR.5q31.3, which is represented by the homozygous SNP combination A(dbSNP RS ID rs778588—physical position 140007011)-C(dbSNP RS ID rs2563298—physical position 140011315)-C(dbSNP RS ID rs5744448—physical position 140014909)-C(dbSNP RS ID rs2569192—physical position 140015208), was given the value 1 and indicated predominantly non-responders while any other sequence was given the value 0 and indicated predominantly responders as can be seen in FIG. 8.

### Example 9

### ADAMTS9-Genotype

The homozygous sequence of BB-AA-AA-BB-AA-BB-BB-AA for the SNP's rs9820942, rs6780659, rs6445415, rs11721258, rs11707584, rs7652817, rs13079218, rs9819183 of ADAMTS9-Intron at Chr. 3p14.1, which is represented by the homozygous SNP combination G(dbSNP RS ID rs9820942—physical position 64560013)-C(dbSNP RS ID rs6780659—physical position 64595571)-A(dbSNP RS ID rs6445415—physical position 64602006)-T(dbSNP RS ID rs11721258—physical position 64605119)-A(dbSNP RS ID rs11707584—physical position 64612402)-G(dbSNP RS ID rs7652817—physical position 64614313)-T(dbSNP RS ID rs13079218—physical position 64617371)-A(dbSNP RS ID rs9819183—physical position 64620883), was given the value 1 and indicated predominantly non-responders (including 1 false negative result) while any other sequence was given the value 0 and indicated responders as can be seen in FIG. 9.

### Example 10

### KLHDC8A-Genotype

The homozygous sequence of AA-BB-AA-BB for the SNP's rs778588, rs2563298, rs5744448, rs2569192 of KLHDC8A-Intron at Chr. 1q32.1, which is represented by the homozygous SNP combination C(dbSNP RS ID rs7549293—physical position 205312280)-T(dbSNP RS ID rs10751436—physical position 205318524)-A(dbSNP RS ID rs913723—physical position 205318854)-T(dbSNP RS ID rs913722—physical position 205318983), was given the value 1 and indicated non-responders while any other sequence was given the value 0 and predominantly indicated responders (including 1 false positive result) as can be seen in FIG. 10.

### Example 11

### LDA-Score (2P1)

LDA-Score(2P1)=5,173156752*(ADAMTS9 Genotype)+0,0010883751*(IG induced (netto) IFN-γ Genex)−2,5111538246.
A LDA-Score(2P1)≦0.0 indicates responders while a LDA-Score(2P1)>0.0 predominantly indicates non-responders (including 1 false negative result) as depictured in FIG. 11.

### Example 12

### LDA-Score (2P2)

LDA-Score(2P2)=5,1584234532*(ADAMTS9 Genotype)+0,0009843151*(IG/LPS induced (netto) IFN-γ Genex)−2,5250980052.
A LDA-Score(2P2)≦0.0 indicates responders while a LDA-Score(2P2)>0.0 predominantly indicates non-responders (including 1 false negative result) as depictured in FIG. 12.

### Example 13

### LDA-Score (2P3)

LDA-Score(2P3)=5,1710817023*(ADAMTS9 Genotype)−0,0526409406*(IG induced (netto) IL-10 release)−2,5189275627.
A LDA-Score(2P3)≦0.0 indicates responders while a LDA-Score(2P3)>0.0 predominantly indicates non-responders (including 1 false negative result) as depictured in FIG. 13.

### Example 14

### LDA-Score (2P4)

LDA-Score(2P4)=5,1547784757*(ADAMTS9 Genotype)+0,0006613541*(IG induced (netto) CCL8 release)−2,5483009377.
A LDA-Score(2P4)≦0.0 indicates responders while a LDA-Score(2P4)>0.0 predominantly indicates non-responders (including 1 false negative result) as depictured in FIG. 14.

### Example 15

### LDA-Score (2P5)

LDA-Score(2P5)=12,6661481683*(ADAMTS9 Genotype)−0,0018215212*(IG induced (netto) CCL20 release)−5,2193484355.
A LDA-Score(2P5)≦0.0 indicates responders while a LDA-Score(2P5)>0.0 indicates non-responders as depictured in FIG. 15.

### Example 16

### LDA-Score (2P6)

LDA-Score(2P6)=5,6030684062*(KLHDC8A Genotype)+0,5886545649*(IG induced (netto) IL-6R release)−2,0540283735.
A LDA-Score(2P6)≦0.0 predominantly indicates responders (including 1 false positive result) while a LDA-Score(2P6)>0.0 indicates non-responders as depictured in FIG. 16.

### Example 17

### LDA-Score (2P7)

LDA-Score(2P7)=6,4011642693*(KLHDC8A Genotype)+0,1385143298*(IG induced (netto) ICAM1 release)−2,870032934.
A LDA-Score(2P7)≦−1.0 indicates responders while a LDA-Score(2P7)>−1.0 indicates non-responders as depictured in FIG. 17.

### Example 18

### LDA-Score (2P8)

LDA-Score(2P8)=5,7296431439*(KLHDC8A Genotype)−0,3050588266*(IG induced (netto) CD32b Genex)−2,8435304986.
A LDA-Score(2P8)≦−1.0 indicates responders while a LDA-Score(2P8)>−1.0 indicates non-responders as depictured in FIG. 18.

### Example 19

### LDA-Score (4P1)

LDA-Score(4P1)=31,5741470438*(ADAMTS9 Genotype)−0,0052245002*(IG induced (netto) CCL20 release)+0,0166330872*(IG induced (netto) CCL8 release)−0,0109678784*(IG/LPS induced (netto) IFN-γ Genex)−13,8885092449.
A LDA-Score(4P1)≦0.0 indicates responders while a LDA-Score(4P1)>0.0 indicates non-responders as depictured in FIG. 19.

### Example 20

### LDA-Score (4P2)

LDA-Score(4P2)=7,0639539622*(KLHDC8A Genotype)−0,2539770554*(IG/LPS induced (netto) CD32b Genex)+0,4613873178*(IG induced (netto) IL-6R release)+0,111066766*(IG induced (netto) ICAM1 release)−3,3328149764.
A LDA-Score(4P2)≦0.0 indicates responders while a LDA-Score(4P2)>0.0 indicates non-responders as depictured in FIG. 20.

This method allows the identification of persons responding/non-responding to any immunoglobulin product suitable for in vivo use such as those applied intravenously, subcutaneously, intramuscularly, ocularly, intrathecially, orally, topically or inhalably for diseases which are in principle accessible to immunoglobulin treatment, such as immune mediated inflammatory diseases, autoimmune diseases, allergies, graft-versus-host reactions and prevention of transplant rejection; any kind of multiple sclerosis or any other demyelinating neurological disease; or relapsing-remitting multiple sclerosis.

The method also permits to predict the probability of a relapse of a MS patient and/or the rate of progression of the disease in terms of disability and or functioning of the patient as measured by clinical scales such as, but not limited to, the expanded disability status scale (EDSS), in particular lupus erythematosus, rheumatoid arthritis or intestinal/bowel diseases such as Crohn's disease, myositis or recurrent abortion.

This method can additionally be used for facilitating the approval or recommendation of immunoglobulins by health authorities for the treatment of any kind of multiple sclerosis or any other demyelinating disease or Lupus erythematosus, rheumatoid arthritis or intestinal/bowel diseases such as Crohn's disease, myositis or recurrent abortion.

TABLE 1

| | | | | SEQ | |
|---|---|---|---|---|---|
| | | | | ID | Strand vs. |
| Gene Symbol | Physical Position | dbSNP RS ID | Flank | No | dbSNP |
| ADAMTS9 | 64560013 | rs9820942 | GCTCAGGAGCTATTAG[A/G]ATTTTTTTTGTATCGA | 1 | same |
| ADAMTS9 | 64595571 | rs6780659 | ACTACAGTGATCAAAA[C/T]AGTCTACTGGCATAAG | 2 | same |
| ADAMTS9 | 64602006 | rs6445415 | TAAAATCTAATTTTAA[A/G]CCAACATATCCATGTG | 3 | same |
| ADAMTS9 | 64605119 | rs11721258 | CGGAAATCTAAATCTG[C/T]ACCTAGATCCTGTGCT | 4 | reverse |
| ADAMTS9 | 64612402 | rs11707584 | TACTTTTATAAGCCCA[A/G]GAAAGATCCCAACCCA | 5 | same |
| ADAMTS9 | 64614313 | rs7652817 | TTAATTGTCTTATAAA[A/G]TTGTCACTCATTTGGA | 6 | same |
| ADAMTS9 | 64617371 | rs13079218 | GATGGATAATCCACCA[C/T]AACGAGACTGATTTTT | 7 | same |
| ADAMTS9 | 64620883 | rs9819183 | ATAACAGTATCAGGAC[A/G]AGCTTCTCTAGGAGGG | 8 | reverse |
| KLHDC8A | 205312280 | rs7549293 | CAGATGGAAACCAACA[C/G]GCATTTGCTGTGGGCT | 9 | reverse |
| KLHDC8A | 205318524 | rs10751436 | AGTGGGTATCCATTAA[C/T]GAATGCATCTGTTCAT | 10 | same |
| KLHDC8A | 205318854 | rs913723 | TCATGCTGGTGTTTCA[A/G]GTTTCTGACATTGCTG | 11 | same |
| KLHDC8A | 205318983 | rs913722 | ACGATGAAGGCTATGA[C/T]CTCCAGCCTACGTTTT | 12 | reverse |
| CD14 | 140007011 | rs778588 | GACACTGAGTTTACTA[A/G]ACTACATAAACTGCTT | 13 | reverse |
| CD14 | 140011315 | rs2563298 | AAATGAATGACACGGA[A/C]CCGTTGTTTAAGATTT | 14 | same |
| CD14 | 140014909 | rs5744448 | GGCCCAAGTCTCATAA[A/C]CTCAGTCGTAAAGCTG | 15 | same |
| CD14 | 140015208 | rs2569192 | GTCCCAGGGCTTTCTA[C/G]CAACCCTAGTACTCGG | 16 | same |

| Physical Position | dbSNP RS ID | Allele A/B | SNP Chromosome | Cytoband | Gene Relationship | Probe Set ID |
|---|---|---|---|---|---|---|
| 64560013 | rs9820942 | A/G | 3 | p14.1 | intron | SNP_A-4303621 |
| 64595571 | rs6780659 | C/T | 3 | p14.1 | intron | SNP_A-2083737 |
| 64602006 | rs6445415 | A/G | 3 | p14.1 | intron | SNP_A-8331608 |
| 64605119 | rs11721258 | C/T | 3 | p14.1 | intron | SNP_A-4294272 |
| 64612402 | rs11707584 | A/G | 3 | p14.1 | intron | SNP_A-1837770 |
| 64614313 | rs7652817 | A/G | 3 | p14.1 | intron | SNP_A-8493544 |
| 64617371 | rs13079218 | C/T | 3 | p14.1 | intron | SNP_A-8697971 |
| 64620883 | rs9819183 | A/G | 3 | p14.1 | intron | SNP_A-2127513 |
| 205312280 | rs7549293 | C/G | 1 | q32.1 | intron | SNP_A-2182772 |
| 205318524 | rs10751436 | C/T | 1 | q32.1 | intron | SNP_A-8663719 |
| 205318854 | rs913723 | A/G | 1 | q32.1 | intron | SNP_A-8663720 |
| 205318983 | rs913722 | C/T | 1 | q32.1 | intron | SNP_A-8663721 |
| 140007011 | rs778588 | A/G | 5 | q31.3 | downstream | SNP_A-8685292 |
| 140011315 | rs2563298 | A/C | 5 | q31.3 | 3UTR | SNP_A-8351179 |
| 140014909 | rs5744448 | A/C | 5 | q31.3 | upstream | SNP_A-8407522 |
| 140015208 | rs2569192 | C/G | 5 | q31.3 | upstream | SNP_A-4194489 |

1. A method for determining the likelihood of response of an individual, suffering from a disease, towards immunoglobulin therapy comprising the steps of
providing a sample containing B- and T-lymphocytes, natural killer cells, invariant T-cells and monocytes of the individual;
genotyping of at least one of the polynucleotides of an ADAMTS9-Intron; a KLHDC8A-Intron or of a flanking region of the CD14 gene, and
awarding the value of 1 for the homozygous Single Nucleotide Polymorphism combinations, which suggests that the blood sample stems from a person which will not respond to immunoglobulin treatment,
while awarding the value of 0 for SNP not meeting that criteria, which suggests that the blood sample stems from a person which will respond to immunoglobulin treatment.

2. The method of claim 1 for determining the likelihood of response of an individual, suffering from a disease, towards immunoglobulin therapy comprising the steps of
providing a sample containing B- and T-lymphocytes, natural killer cells, invariant T-cells and monocytes of the individual;
genotyping of the ADAMTS9-Intron at Chr.3p14.1 and dbSNP RS ID's rs9820942, rs6780659, rs6445415, rs11721258, rs11707584, rs7652817, rs13079218, rs9819183 and/or of the KLHDC8A-Intron at Chr.1q32.1 and dbSNP RS ID's rs7549293, rs10751436, rs913723, rs913722 and/or of the CD14 flanking region at Chr.5q31.3 and dbSNP RS ID's rs778588, rs2563298, rs5744448, rs2569192 and awarding the value of 1 for the homozygous SNP (Single Nucleotide Polymorphism) combinations BB-AA-AA-BB-AA-BB-BB-AA of the ADAMTS9-Intron, which is represented by the homozygous SNP combination G(dbSNP RS ID rs9820942—physical position 64560013)-C(dbSNP RS ID rs6780659—physical position 64595571)-A(dbSNP RS ID rs6445415—physical position 64602006)-T(dbSNP RS ID rs11721258—physical position 64605119)-A(dbSNP RS ID rs11707584—physical position 64612402)-G(dbSNP RS ID rs7652817—physical position 64614313)-T(dbSNP RS ID rs13079218—physical position 64617371)-A(dbSNP RS ID rs9819183—physical position 64620883), AA-BB-AA-BB of the KLHDC8A-Intron, which is represented by the homozygous SNP combination C(dbSNP RS ID rs7549293—physical position 205312280)-T(dbSNP RS ID rs10751436—physical position 205318524)-A(dbSNP RS ID rs913723—physical position 205318854)-T(dbSNP RS ID rs913722—physical position 205318983), and AA-BB-BB-AA of the CD14 flanking region at said physical positions, which is represented by the homozygous SNP combination A(dbSNP RS ID rs778588—physical position 140007011)-C(dbSNP RS ID rs2563298—physical position 140011315)-C(dbSNP RS ID rs5744448—physical position 140014909)-C(dbSNP RS ID rs2569192—physical position 140015208) in the same order by the allele combination containing the nucleic acids AA-CC-CC-CC in the relevant position, which suggests that the blood sample stems from a person which will not respond to IG treatment, while awarding the value of 0 for SNP not meeting that criteria, which suggests that the blood sample stems from a person which will respond to IG treatment.

3. The method of claim 1 wherein the genotyping status is complemented with parameters by determination of at least one of the amount of cytokines released from or their expressed genes on cells, wherein cytokines are selected from the group of Interferon-gamma (IFN-γ), Interleukin-8 (CXCL8), C-X-C motif chemokine 10 (CXCL10), chemokine C-C motif ligand 8 (CCL8), chemokine C-C motif ligand 20 (CCL20), Interleukin-10 (IL-10) and Stem cell factor (SCF).

4. The method of claim 1 wherein the genotyping status is complemented with parameters by determination of the amount at least one of the proteins CD32b, CD16, IL-6R (Interleukin-6 receptor) and ICAM-1 (Inter Cellular Adhesion Molecule 1) released from and/or or their expressed genes on cells.

5. The method of claim 3 wherein the release of said proteins and the expression of their genes is determined after ex vivo exposure of samples with immunoglobulin, in particular IgG, IgM, IgA or a combination thereof.

6. The method of claim 1 wherein genotyping, protein release and gene expression are determined in whole blood, blood fractions, cell fractions or plasma.

7. The method of claim 3 wherein a sample is incubated in presence of a stimulant in at least one assay in presence of immunoglobulins and in at least one assay in absence of immunoglobulins as control and wherein the stimulant is selected from the group consisting of lipopolysaccharides (LPS), phorbol-12-myristate-13 acetate PMA)/ionomycin, monoclonal antibodies binding to receptors on leukocytes or combinations thereof.

8. The method according to claim 3 wherein the amount of immunoglobulins used in assays is from about 0.01 to about 100 mg/ml in particular from about 1 to about 50 mg/ml.

9. The method of claim 1 wherein the method is performed before and/or during the treatment of a patient with immunoglobulin.

10. The method of claim 1 wherein the genotyping status of the ADAMTS9-Intron is complemented by the parameter “IG induced (netto) CCL20 release” and the LDA-Score (Linear Discriminant Analysis) determined by incorporation of the value for genotyping status and the value of protein amount given in [pg/ml] into the formula:
LDA−LDA-Score(2P5)=12,6661481683*(ADAMTS9 Genotype)−0,0018215212*(IG induced (netto) CCL20 release)−5,2193484355, wherein a LDA-Score(2P5)≦0.0 indicates responders while a LDA-Score(2P5)>0.0 indicates non-responders.

11. The method of claim 1 wherein the genotyping status of the ADAMTS9-Intron is complemented by the parameter “IG induced (netto) CCL8 release” and the LDA-Score determined by incorporation of the value for genotyping status and the value of protein amount given in [pg/ml] into the formula:
LDA-Score(2P4)=5,1547784757*(ADAMTS9 Genotype) 0,0006613541*(IG induced (netto) CCL8 release)−2,5483009377, wherein a LDA-Score(2P4)≦0.0 indicates responders while a LDA-Score(2P4)>0.0 indicates non-responders.

12. The method of claim 1 wherein the genotyping status of the ADAMTS9-Intron is complemented by the parameter “IG induced (netto) IL-10 release” and the LDA-Score determined by incorporation of the value for genotyping status and the value of protein amount given in [pg/ml] into the formula:
LDA-Score(2P3)=5,1710817023*(ADAMTS9 Genotype) 0,0526409406*(IG induced (netto) IL-10 release)−2,5189275627, wherein a LDA-Score(2P3)≦0.0 indicates responders while a LDA-Score(2P3)>0.0 indicates non-responders.

13. The method of claim 1 wherein the genotyping status of the ADAMTS9-Intron is complemented by the parameter “IG/LPS induced (netto) IFN-γ Genex” and the LDA-Score determined by incorporation of the value for genotyping status and the value of transcript numbers given in [transcripts/μl] into the formula:
LDA-Score(2P2)=5,1584234532*(ADAMTS9 Genotype)+0,0009843151*(IG/LPS induced (netto) IFN-γ Genex)−2,5250980052,
wherein a LDA-Score(2P2)≦0.0 indicates responders while a LDA-Score(2P2)>0.0 indicates non-responders.

14. The method of claim 1 wherein the genotyping status of the ADAMTS9-Intron is complemented by the parameter “IG induced (netto) IFN-γ Genex” and the LDA-Score determined by incorporation of the value for genotyping status and the value of transcript numbers given in [transcripts/μl] into the formula:
LDA-Score(2P1)=5,173156752*(ADAMTS9 Genotype)+0,0010883751*(IG induced (netto) IFN-γ Genex)−2,5111538246,
wherein a LDA-Score(2P1)≦0.0 indicates responders while a LDA-Score(2P1)>0.0 indicates non-responders.

15. The method of claim 1 wherein the genotyping status of the ADAMTS9-Intron is complemented by the parameters “IG induced (netto) CCL20 release” and “IG induced (netto) CCL8 release” and the LDA-Score determined by incorporation of the value for genotyping status and the values of transcript numbers and protein amount given in [transcripts/μl] and [pg/ml] into the formula:
LDA-Score(3P2)=28,427707664*(ADAMTS9 Genotype)−0,0046972337*(IG induced (netto) CCL20 release)+0,0129144727*(IG induced (netto) CCL8 release)−12,7163079623,
wherein a LDA-Score(3P2)≦0.0 indicates responders while a LDA-Score(3P2)>0.0 indicates non-responders.

16. The method of claim 1 wherein the genotyping status of the ADAMTS9-Intron is complemented by the parameters “IG induced (netto) CCL20 release”, “IG induced (netto) CCL8 release”, and “IG/LPS induced (netto) IFN-γ Genex” and the LDA-Score determined by incorporation of the value for genotyping status and the values of transcript numbers and protein amount given in [transcripts/μl] and [pg/ml] into the formula:
LDA-Score(4P1)=31,5741470438*(ADAMTS9 Genotype)−0,0052245002*(IG induced (netto) CCL20 release)+0,0166330872*(IG induced (netto) CCL8 release)−0,0109678784*(1G/LPS induced (netto) IFN-γ Genex)−13,8885092449,
wherein a LDA-Score(4P1)≦0.0 indicates responders while a LDA-Score(4P1)>0.0 indicates non-responders.

17. The method of claim 1 wherein the genotyping status of the ADAMTS9-Intron is complemented by the parameters “IG induced (netto) CCL20 release”, “IG induced (netto) CCL8 release”, “IG induced (netto) CXCL8 release” and “IG/LPS induced (netto) IFN-γ Genex” and the LDA-Score determined by incorporation of the value for genotyping status and the values of transcript numbers and protein amount given in [transcripts/μl] and [pg/ml] into the formula:
LDA-Score(5P1)=107,2468831*(ADAMTS9 Genotype)−0.038780771*(1G/LPS induced (netto) IFN-γ Genex)−0.017866668*(1G induced (netto) CCL20 release)+0.044172208*(IG induced (netto) CCL8 release)+0.002477736*(IG induced (netto) CXCL8 release)−47.9651381, wherein a LDA-Score(5P1)≦0.0 indicates responders while a LDA-Score(5P1)>0.0 indicates non-responders.

18. The method of claim 1 wherein the genotyping status of the ADAMTS9-Intron is complemented by the parameters “IG induced (netto) CCL20 release”, “IG induced (netto) CCL8 release”, “IG induced (netto) SCF release” and “IG/LPS induced (netto) IFN-γ Genex” and the LDA-Score determined by incorporation of the value for genotyping status and the values of transcript numbers and protein amount given in [transcripts/μl] and [pg/ml] into the formula:
LDA-Score(5P2)=89.56250541*(ADAMTS9 Genotype)−0.128146913*(1G/LPS induced (netto) IFN-γ Genex)−0.015495947*(IG induced (netto) CCL20 release)+0.058499044*(IG induced (netto) CCL8 release)+0.008472595*(IG induced (netto) SCF release)−43.33685048,
wherein a LDA-Score(5P2)≦0.0 indicates responders while a LDA-Score(5P2)>0.0 indicates non-responders.

19. The method of claim 1 wherein the genotyping status of the ADAMTS9-Intron is complemented by the parameters “IG induced (netto) CCL20 release”, “IG induced (netto) CCL8 release”, “IG induced (netto) CXCL10 release”, “IG induced (netto) IL-10 release”, “IG induced (netto) CXCL8 release”, “IG induced (netto) ICAM1 Genex”, “IG/LPS induced (netto) IFN-γ Genex” and “IG induced (netto) CXCL8 Genex” and the LDA-Score determined by incorporation of the value for genotyping status and the values of transcript numbers and protein amount given in [transcripts/μl] and [pg/ml] into the formula:
LDA-Score(9P)=108,5705785*(ADAMTS9 Genotype)−0.065661811*(IG induced (netto) ICAM1 Genex)−0.14179279*(IG induced (netto) IFN-γ Genex)−0.00521369*(IG induced (netto) CXCL8 Genex)−0.017983675*(IG induced (netto) CCL20 release)+0.018722767*(IG induced (netto) CCL8 release)+0.001625748*(IG induced (netto) CXCL10 release)+0.425763386*(IG induced (netto) IL-10 release)+0.004389251*(IG induced (netto) CXCL8 release)−48.34366669,
wherein a LDA-Score(9P)≦0.0 indicates responders while a LDA-Score(9P)>0.0 indicates non-responders.

20. The method of claim 1 wherein the genotyping status of the KLHDC8A-Intron is complemented by the parameter “IG induced (netto) IL-6R release” and the LDA-Score determined by incorporation of the value for genotyping status and the value of protein amount given in [pg/ml] into the formula:
LDA-Score(2P6)=5,6030684062*(KLHDC8A Genotype) 0,5886545649*(IG induced (netto) IL-6R release)−2,0540283735,
wherein a LDA-Score(2P6)≦0.0 indicates responders while a LDA-Score(2P6)>0.0 indicates non-responders.

21. The method of claim 1 wherein the genotyping status of the KLHDC8A-Intron is complemented by the parameter “IG induced (netto) ICAM1 release” and the LDA-Score determined by incorporation of the value for genotyping status and the value of protein amount given in [pg/ml] into the formula:
LDA-Score(2P7)=6,4011642693*(KLHDC8A Genotype)+0,1385143298*(IG induced (netto) ICAM1 release)−2,870032934,
wherein a LDA-Score(2P7)≦−1.0 indicates responders while a LDA-Score(2P7)>−1.0 indicates non-responders.

22. The method of claim 1 wherein the genotyping status of the KLHDC8A-Intron is complemented by the parameter “IG induced (netto) CD32b Genex” and the LDA-Score determined by incorporation of the value for genotyping status and the values of transcript numbers given in [transcripts/μl] into the formula:
LDA-Score(2P8)=5,7296431439*(KLHDC8A Genotype)−0,3050588266*(IG induced (netto) CD32b Genex)−2,8435304986,
wherein a LDA-Score(2P8)≦−1.0 indicates responders while a LDA-Score(2P8)>−1.0 indicates non-responders.

23. The method of claim 1 wherein the genotyping status of the KLHDC8A-Intron is complemented by the parameters “IG/LPS induced (netto) CD16 Genex” and “IG induced (netto) ICAM-1 release” and the LDA-Score determined by incorporation of the value for genotyping status and the values of transcript numbers and protein amount given in [transcripts/μl] and [pg/ml] into the formula:
LDA-Score(3P1)=6,207662683*(KLHDC8A Genotype)−0.007323378*(IG/LPS induced (netto) CD16 Genex)+0.219033761*(IG induced (netto) ICAM-1 release)−5.456170752,
wherein a LDA-Score(3P1)≦−1.0 indicates responders while a LDA-Score(3P1)>−1.0 indicates non-responders.

24. The method of claim 1 wherein the genotyping status of the KLHDC8A-Intron is complemented by the parameters “IG/LPS induced (netto) CD32b Genex”, “IG induced (netto) IL-6R release” and “IG induced (netto) ICAM1 release” and the LDA-Score determined by incorporation of the value for genotyping status and the values of transcript numbers and protein amount given in [transcripts/μl] and [pg/ml] into the formula:
LDA-Score(4P2)=7,0639539622*(KLHDC8A Genotype)−0,2539770554*(1G/LPS induced (netto) CD32b Genex)+0,4613873178*(IG induced (netto) IL-6R release)+0,111066766*(IG induced (netto) ICAM1 release)−3,3328149764,
wherein a LDA-Score(4P2)≦0.0 indicates responders while a LDA-Score(3P1)>0.0 indicates non-responders.

25. The method of claim 1 wherein the genotyping status of the KLHDC8A-Intron is complemented by the parameters “IG/LPS induced (netto) CD16 Genex”, “IG/LPS induced (netto) CD32b Genex”, “IG induced (netto) ICAM-1 release” and “IG induced (netto) IL-6R release” and the LDA-Score determined by incorporation of the value for genotyping status and the values of transcript numbers and protein amount given in [transcripts/μl] and [pg/ml] into the formula:
LDA-Score(5P3)=11.44098342*(KLHDC8A Genotype)−0.045599133*(1G/LPS induced (netto) CD16 Genex)−0.535989358*(1G/LPS induced (netto) CD32b Genex)+0.225465018*(IG induced (netto) ICAM-1 release)+3.14495298*(IG induced (netto) IL-6R release)−3.9398568,
wherein a LDA-Score(5P3)≦0.0 indicates responders while a LDA-Score(5P3)>0.0 indicates non-responders.

26. The method of claim 1 wherein the genotyping status of the KLHDC8A-Intron is complemented by the parameters “IG induced (netto) CD32b Genex”, “IG induced (netto) ICAM-1 Genex”, “IG/LPS induced (netto) CD32b Genex” and “IG induced (netto) IL-6R release” and the LDA-Score determined by incorporation of the value for genotyping status and the values of transcript numbers and protein amount given in [transcripts/μl] and [pg/ml] into the formula:
LDA-Score(5P4)=9.476844721*(KLHDC8A Genotype)−0.361944446*(IG induced (netto) CD32b Genex)+0.008332887*(IG induced (netto) ICAM-1 Genex)−0.939416614*(IG/LPS induced (netto) CD32b Genex)+0,951418988*(IG induced (netto) IL-6R release)−4.232325519,
wherein a LDA-Score(5P4)≦0.0 indicates responders while a LDA-Score(5P4)>0.0 indicates non-responders.

27. The method of claim 1 wherein the indication of responder or non-responder is confirmed by at least one additional, but different method of claim 1.

28. The method of claim 1 wherein any immunoglobulin product suitable for in vivo use is concerned such as those applied intravenously, subcutaneously, intramuscularly, ocularly, intrathecially, orally, topically or inhalably.

29. The method according to claim 1 wherein the disease is selected from the group consisting of inflammatory mediated immune diseases, autoimmune diseases, allergies, graft-versus-host reactions and prevention of transplant rejection; any kind of multiple sclerosis or any other demyelinating neurological disease; or relapsing-remitting multiple sclerosis.

30. The method according to claim 1 permitting to predict the probability of a relapse of a MS patient and/or the rate of progression of the disease in terms of disability and or functioning of the patient as measured by clinical scales such as, but not limited to, the expanded disability status scale (EDSS), in particular lupus erythematosus, rheumatoid arthritis or intestinal/bowel diseases such as Crohn's disease, myositis or recurrent abortion.

31. Use of the method of claim 1 for facilitating the approval or recommendation of immunoglobulins by health authorities for the treatment of any kind of multiple sclerosis or any other demyelinating disease or Lupus erythematosus, rheumatoid arthritis or intestinal/bowel diseases such as Crohn's disease, myositis or recurrent abortion.


SEQUENCE LISTING (16 sequences)

SEQ ID NO: 1 - DNA, 33 bp, human
gctcaggagctattagrattttttttgtatcga

SEQ ID NO: 2 - DNA, 33 bp, human
actacagtgatcaaaayagtctactggcataag

SEQ ID NO: 3 - DNA, 33 bp, human
taaaatctaattttaarccaacatatccatgtg

SEQ ID NO: 4 - DNA, 33 bp, human
cggaaatctaaatctgyacctagatcctgtgct

SEQ ID NO: 5 - DNA, 33 bp, human
tacttttataagcccargaaagatcccaaccca

SEQ ID NO: 6 - DNA, 33 bp, human
ttaattgtcttataaarttgtcactcatttgga

SEQ ID NO: 7 - DNA, 33 bp, human
gatggataatccaccayaacgagactgattttt

SEQ ID NO: 8 - DNA, 33 bp, human
ataacagtatcaggacragcttctctaggaggg

SEQ ID NO: 9 - DNA, 33 bp, human
cagatggaaaccaacasgcatttgctgtgggct

SEQ ID NO: 10 - DNA, 33 bp, human
agtgggtatccattaaygaatgcatctgttcat

SEQ ID NO: 11 - DNA, 33 bp, human
tcatgctggtgtttcargtttctgacattgctg

SEQ ID NO: 12 - DNA, 33 bp, human
acgatgaaggctatgayctccagcctacgtttt

SEQ ID NO: 13 - DNA, 33 bp, human
gacactgagtttactaractacataaactgctt

SEQ ID NO: 14 - DNA, 33 bp, human
aaatgaatgacacggamccgttgtttaagattt

SEQ ID NO: 15 - DNA, 33 bp, human
ggcccaagtctcataamctcagtcgtaaagctg

SEQ ID NO: 16 - DNA, 33 bp, human
gtcccagggctttctascaaccctagtactcgg